(19) United States
(12) Patent Application Publication
(10) Pub. No.: US 2022/0220429 A1
(43) Pub. Date: Jul. 14, 2022

(54) SYSTEM AND METHOD FOR METHANE BIODEGRADATION

(71) Applicant: PARSONS CORPORATION, Centreville, VA (US)

(72) Inventor: Glenn Ulrich, Centreville, VA (US)

(73) Assignee: PARSONS CORPORATION, Centreville, VA (US)

(21) Appl. No.: 17/647,655

(22) Filed: Jan. 11, 2022

Related U.S. Application Data
(60) Provisional application No. 63/137,550, filed on Jan. 14, 2021.

(51) Int. Cl. C12M 1/00; C12M 1/12; C12M 1/42; C12M 1/34; C12N 1/20; C12N 1/30; B01D 53/85; B01D 53/72; B09C 1/10

(52) U.S. Cl. CPC C12M 23/18; C12M 25/14; C12M 35/08; C12M 41/26; C12M 43/00; B09C 2101/00; C12N 1/30; B01D 53/85; B01D 53/72; B09C 1/10; B01D 2251/95; C12N 1/20

(57) ABSTRACT

Biodegradation media placed in, around, and/or above a methane source reduces the quantum of methane and other alkane gases such as ethane, propane, and butane released into the atmosphere under diverse and fluctuating environmental conditions over a sustainable and/or extended duration. Non-biodegradable material configured for methane biodegradation possesses enhanced drainage of precipitation, improved gas transmission and gas exchange, moisture retention, and a nutrient sustainability.

### RELATED APPLICATION

The present application relates to and claims the benefit of priority to United States Provisional Patent Application No. 63/137,550 filed 14 Jan. 2021 which is hereby incorporated by reference in its entirety for all purposes as if fully set forth herein.

### BACKGROUND OF THE INVENTION

### Field of the Invention

Embodiments of the present invention relate, in general, to methane mitigation and more particularly to sustainable aerobic methane biodegradation.

### Relevant Background

Gases that trap heat in the atmosphere are called greenhouse gases. One such gas is Methane (CH4). Methane is emitted during the production of coal, natural gas, and oil as well as from livestock and other agricultural practices, land use and by the decay of organic waste in municipal solid waste landfills. Globally, 50-65 percent of total CH4 emissions come from human activities.

In 2019, methane (CH4) accounted for about 10 percent of all U.S. greenhouse gas emissions from human activities. Human activities emitting methane include leaks from oil wells, natural gas systems, raising of livestock and landfills. Natural gas and petroleum systems account for approximately 30% of CH4 emissions in the United States.

Natural processes in soil and chemical reactions in the atmosphere help remove naturally occurring CH4 from the atmosphere. But the human activities mentioned above have strained and/or exceeded nature's ability to mitigate methane in our atmosphere. While methane's lifetime in the atmosphere is much shorter than carbon dioxide (CO2), CH4 is more efficient at trapping radiation than CO2. Pound for pound, the comparative impact of CH4 is 25 times greater than CO2 over a 100-year period.

There are tens of thousands of oil and gas wells, livestock sites, landfills, pipelines, and methane seeps (natural and anthropogenic) leaking natural gas around the world, contributing to increasing atmospheric concentrations of methane. For example, natural gas (of which the primary component is typically methane) can leak directly through wells or through subsurface soil in the vicinity of leaking wells. Wells (natural gas and oil), landfills, and other sources may leak indefinitely.

As the natural gas production rates in producing gas fields decrease to levels that can't be economically produced or utilized, the wells are often plugged or flared (burning). Plugging wells is costly and often not a permanent solution and flares have been associated with significant methane emissions. Similarly, as methanogenesis and methane production rates in landfills decrease, the gas can no longer be economically utilized. To prevent over pressurization the gas is vented to the atmosphere or flared leading to methane emissions and emissions of harmful byproducts generated from flaring.

Methane consuming biocovers comprised of thin layers of organic materials such as wood chips, compost, and mulch have been placed over methane seeps on top of landfills (J. Streese and R. Stegmann. 2003; I. Pecorini and R. Iannelli. 2020). There are several limitations to organic biocovers including oxygen utilization attributed to the biodegradation of the organic material diverting oxygen from methane oxidation and deterioration of the organic biocover media structure over time. (B. Y. Sadasivam and K. R. Reddy, 2014). Furthermore, biocovers are susceptible to freezing during cold conditions, desiccation during dry conditions, and saturation during wet conditions all of which will inhibit methane biodegradation. Finally, biocovers are not applicable to localized areas of methane leaking at a high rate.

What is needed, therefore, is a sustainable methane biodegradation media, with the capability of operating over long periods of time (decades or longer) with limited or no maintenance under diverse and fluctuating environmental conditions, and a methodology to apply such media to eliminate or at least mitigate human activity generated methane. These and other deficiencies of the prior art are addressed by one or more embodiments of the present invention.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### SUMMARY OF THE INVENTION

Biodegradation media placed in, around, and/or above a methane source reduces the quantum of methane and other alkane gases such as ethane, propane, and butane released into the atmosphere under diverse and fluctuating environmental conditions over a sustainable and/or extended duration. Non-biodegradable material configured for methane biodegradation comprise media characterized as having (a) enhanced drainage of precipitation, (b) improved gas transmission and gas exchange, (c) moisture retention, and (d) a long-lasting nutrient source.

One component of a sustainable aerobic methane biodegradation media includes methanotrophs native to the environment near the methane source. These methanotrophs are combined with an inorganic matrix enabling sustained methane biodegradation. The matrix further includes a structure configured to manage moisture and create a sustainable methane biodegradation environment.

The methane biodegradation media outlined above includes methanotrophic bacteria based on native soil extracted at the methane source. In one version of the present invention the media includes methanotrophs replicated from the native soil at the methane source. These and other methanotrophs are uniformly dispersed throughout the matrix aiding sustainable methane biodegradation.

As mentioned above, the inorganic matrix of the present invention is configured to manage moisture and create a sustainable methane biodegradation environment. In one version of the present invention the inorganic matrix includes a moisture management component configured to manage moisture content and a drainage component configured to manage infiltrating precipitation. The moisture management component can be selected from the group consisting of vermiculate, peat, perlite, and sawdust while the drainage component can be selected from the group consisting of pumice, sand, perlite, and gravel. Concentrations and ratios of the drainage component and the moisture component, indeed the entire composition of the inorganic matrix, is based on a methane flow rate at the methane source and the environment in which the methane source exists.

For example, the thickness and size (how much matrix the methane must flow through to be mitigated) of the inorganic matrix is based on the methane flow rate. In other embodiments of the present invention the structure of the matrix can be varied based on the environment including a vertical layered structure having one or more successive layers of the moisture management component and the drainage component. In an arid environment, the inorganic matrix structure can include a base layer of a highly concentrated layer of a moisture retaining material selected from a group consisting of vermiculate, peat, perlite, and sawdust. By comparison, in a tropical environment, the inorganic matrix structure may include a sloped drainage layer of a material configured to manage infiltrating precipitation selected from the group consisting of pumice, sand, perlite, and gravel. Ratios, concentrations, and structures of the moisture management component versus the drainage component vary based on the environment and methane flow rate.

In another embodiment of the present invention, the moisture management component includes materials with a plurality of cation exchange sites. These cation exchange sites are bound with added nutrient cations. The matrix can also include magnesium and/or calcium configured to block nutrient uptake by the cation exchange sites. Added cationic nutrients sufficient for methane biodegradation are also part of the matrix composition.

In one embodiment of the present invention a methodology applies biodegradation media to a source of methane and other alkane gasses (including but not limited to landfills and natural gas vent pipes) reducing the quantum of methane released into the atmosphere under diverse and fluctuating environmental conditions over a sustainable and/or extended duration. Methanotrophs native to a methane source are seeded into a methane biodegradation media. The non-biodegradable media is interposed between the methane source and the surface in formats enabling sustained aerobic methane biodegradation.

One method for sustained aerobic methane biodegradation includes identifying, at a methane source site, methanotrophs. At the methane source site, a sufficiency of methanotrophs for methane biodegradation is determined considering the methane flow rate. Having such information in hand, the process for methane biodegradation continues by configuring, at the methane source site, an inorganic matrix for sustained methane biodegradation wherein the inorganic matrix includes an inorganic matrix structure configured to manage moisture content, manage moisture drainage, and create a sustainable methane biodegradation environment.

The methodology includes, in one version of the present invention, extracting methanotrophs from the methane source site and thereafter growing methanotrophic bacteria. Methanotrophs at the methane source site can then be supplemented with grown methanotrophic bacteria.

One aspect of the method for sustained aerobic methane biodegradation, according to the present invention, includes configuring the inorganic matrix structure to provide sufficient oxygen diffusion into the inorganic matrix and surface area interaction between methanotrophs, methane, and oxygen based on the methane flow rate. Layering the inorganic matrix structure vertically having one or more successive layers of the moisture management component and the drainage component, is another feature the method for sustained aerobic methane biodegradation as is configuring the matrix for arid or tropical environments.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 is a graphic depiction of methanogenesis, and methane oxidation as would be known to one of reasonable skill in the relevant art;

FIG. 2 shows a sustainable aerobic methane biodegradation media imposed over a manmade methane source according to one embodiment of the present invention;

FIG. 3A presents a graphical representation of increased methane biodegradation based on amended methanotrophs inoculum;

FIG. 3B presents a graphical representation production rates of methane biodegradation according to one embodiment of the present invention based various matrix configurations;

FIG. 4 presents one version of a layered structure for a sustainable aerobic methane biodegradation media according to one embodiment of the present invention;

FIG. 5 presents another version of a layered structure for a sustainable aerobic methane biodegradation media according to one embodiment of the present invention;

FIG. 6 presents a mounding structure with augmented oxidation according to one embodiment of the present invention; and

FIGS. 7A-7F present a flowchart of a methodology for sustained aerobic methane biodegradation according to one embodiment of the present invention.

The Figures depict embodiments of the present invention for purposes of illustration only. Like numbers refer to like elements throughout. In the figures, the sizes of certain lines, layers, components, elements, or features may be exaggerated for clarity.

One skilled in the art will readily recognize from the following discussion that alternative embodiments of the structures and methods illustrated herein may be employed without departing from the principles of the invention described herein.

### DESCRIPTION OF THE INVENTION

A sustainable methane biodegradation media reduces the quantum of methane under diverse and fluctuating environmental conditions and/or for an extended duration. Methanotrophs native to a methane source are, in one embodiment, identified, extracted, replicated, and seeded into a biodegradation media. The non-biodegradable media, comprised of methanotrophs and an inorganic matrix and characterized as having (a) enhanced drainage of precipitation, (b) moisture retention, and (c) long-lasting sources of nutrients, is interposed between a methane source and the surface enabling sustained aerobic methane biodegradation thereby reducing methane released into the atmosphere.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims are not limited to the bibliographical meanings but are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “Methanogenesis” it is meant an anaerobic respiration that generates methane as the final product of metabolism. It is the formation of methane by microorganisms known as methanogens.

By the term “Methanotrophs” (sometimes called methanophiles) it is meant prokaryotes that metabolize methane as their source of carbon to unlock the energy of oxygen, nitrate, sulfate, or other oxidized species. Methanotrophs are bacteria or archaea, can grow aerobically or anaerobically, and require single-carbon compounds to survive. Methanotrophs are especially common in or near environments where methane is produced, although some methanotrophs can oxidize atmospheric methane. Their habitats include wetlands, soils, marshes, rice paddies, landfills, aquatic systems (lakes, oceans, streams) and more. In functional terms, methanotrophs are referred to as methane-oxidizing bacteria. However, methane-oxidizing bacteria encompass other organisms that are not regarded as sole methanotrophs. For this reason, methane-oxidizing bacteria have been separated into subgroups: methane-assimilating bacteria (MAB) groups, the methanotrophs, and autotrophic ammonia-oxidizing bacteria (AAOB), which co-oxidize methane.

By the term “methane oxidization” is meant a microbial metabolic process for energy generation and carbon assimilation from methane that is carried out by specific groups of bacteria, the methanotrophs. Methane (CH4) is oxidized with molecular oxygen (O2) to carbon dioxide (CO2).

By the term “biodegradation” is meant the degradation of the materials into environmentally acceptable products such as water, carbon dioxide, and biomass.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

The terminology used herein is for the purpose of describing embodiments only and is not intended to be limiting of the invention. As used herein, the singular forms “a,” “an” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted” etc., another element, it can be directly on, attached to, connected to, coupled with, or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

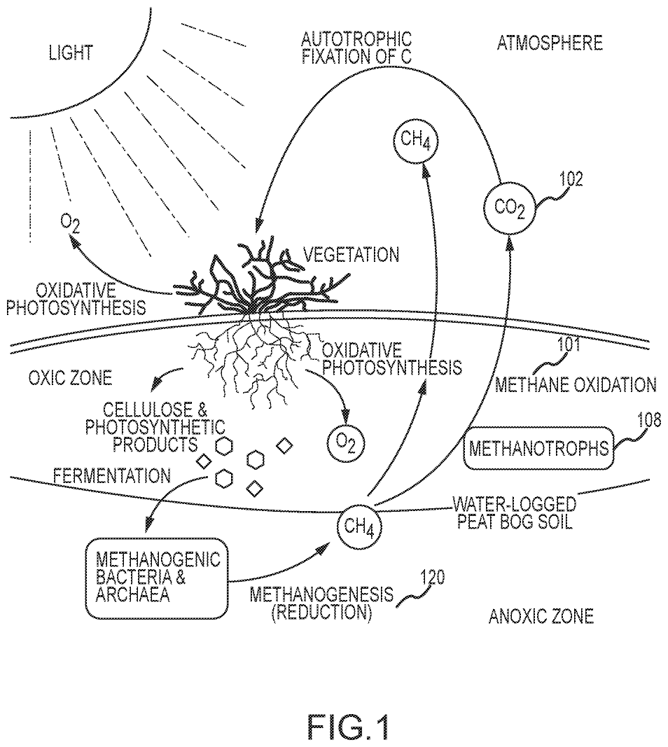

As shown in FIG. 1, Methanotrophs 108 play an important role in the oxidation of methane in the natural environment. Methanotrophs (sometimes called methanophiles) are prokaryotes that metabolize methane as their source of carbon to unlock the energy of oxygen, nitrate, sulfate, or other oxidized species. They are bacteria, can grow aerobically or anaerobically, and require single-carbon compounds to survive. Functionally, methanotrophs 108 are methane-oxidizing bacteria. Under aerobic conditions, methanotrophs combine oxygen and methane to form formaldehyde, which is then incorporated into organic compounds via the serine pathway or the ribulose monophosphate (RuMP) pathway, resulting in the release of carbon dioxide 102.

Under certain conditions, aerobic methane biodegradation 101 happens naturally. Rice paddies, mud pots, peatlands, marshes, and the like are common environments in which aerobic methane biodegradation (methane oxidation) 101 occurs naturally as methane filtrates to the surface after methanogenesis 120.

Mitigation of manmade methane sources is accomplished, according to one embodiment of the present invention, by creating a localized aerobic methane biodegradation environment. As previously described, methane source locations are numerous and many of them are man-made. Landfills and oil wells are two primary sources of methane. In a landfill buried trash includes methanogens which generate methane as part of the decomposition process. Oil and gas wells provide a pathway by which methane, otherwise trapped, or forced to find a path likely through a methanotrophic rich environment, is freely released.

As each methane source is unique, and the conditions surrounding the methane source change seasonally, with individual precipitation events, and over long periods of time, the present invention creates a sustainable methane biodegradation environment that is configured to oxidize methane independent of these varied conditions. While methanotrophs are necessary for aerobic biodegradation of methane, not all methanotrophs are created equal. While one methanotrophic bacteria may be very efficient at methane biodegradation under laboratory conditions, it may not function as well under actual field conditions, nor may it be sustainable.

The present invention uses methanotrophs native to the soil at a methane source location as the basis by which to mitigate methane. One of reasonable skill in the relevant art will appreciate that microorganisms or bacteria that oxidize methane under aerobic conditions differ from one ecosystem to another. A methanotroph which thrives in a peatbog in a tropical climate likely has little sustainability in a dessert environment. Similarly, methanotrophs that have adapted to an arid climate may be eradicated when introduced to a wetland environment. One aspect of the present invention is its ability to adapt to the environment in which the methane source is identified. Not only is the matrix composition varied, but the methanotrophs native to the local environment are identified and when necessary supplemented in kind. Moreover, a sustainable methane biodegradation environment is created at the methane source.

In one embodiment of the present invention an inorganic solid-phase matrix maximizes contact between methane and methanotrophs. The matrix can include soil from the area of the natural methane leak (“Native Soil”), fine sand, medium sand, coarse sand, pumice of various particle sizes, crushed limestone, vermiculite, perlite, and the like (the “Solid Phase Matrix Materials”).

Inorganic material including perlite, vermiculite, pumice, and the like are combined at concentrations and ratios in a particular configuration suited for a specific environment. For example, in a wet environment the matrix can comprise a highly concentrated layer of a drainage component, such as perlite, to facilitate drainage of infiltrating precipitation and promote gas distribution within the matrix. Similarly, arid environments can require a higher concentration and/or ratio of a moisture management component such as vermiculite to retain moisture in support of methane oxidation.

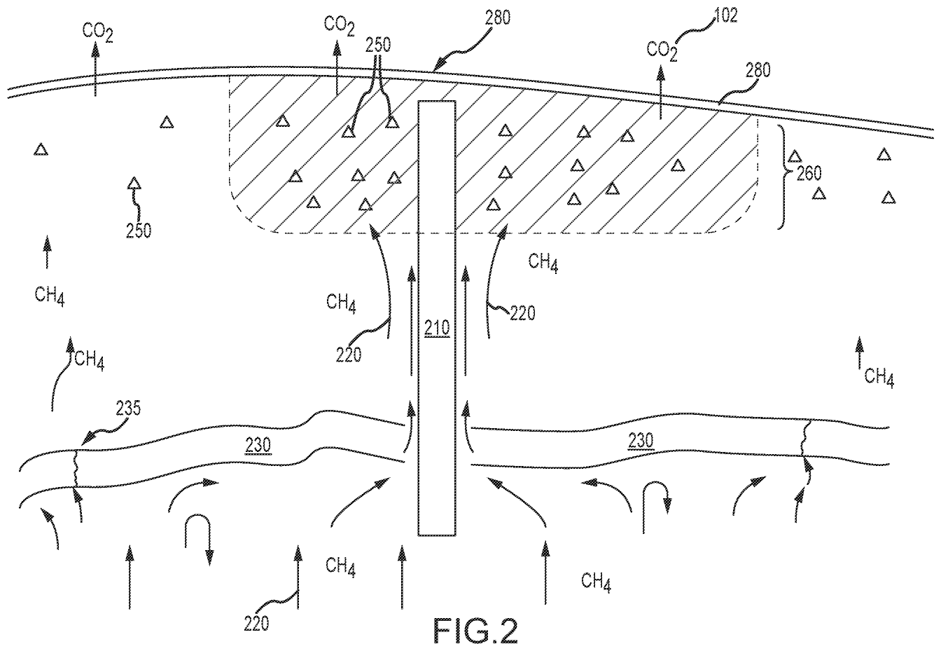

FIG. 2 depicts, at a high-level implementation of a sustainable aerobic methane degradation media 260. In the scenario shown in the FIG. 2, a well 210 or column of some sort has provided a pathway by which methane 220 can migrate to the surface and ultimately into the atmosphere. In this example methane is present below the surface and constrained from migration due a natural barrier 230. The barrier may be nonporous rock or clay or other substances that inhibit the free transmission of methane and other gases. In many instances the barrier is not impervious. Indeed, paths 235 may exist by which methane may escape toward the surface. However, the flow rate of such methane is low and/or dispersed and generally mitigated by methanotrophs 250 native in the soil.

The manmade column 260 or well piercing the barrier 230 provides an unnatural path for the methane to migrate to the surface. The methane flow rate at and near the column, or methane source, is substantially larger than what would otherwise be naturally occurring.

According to one embodiment of the present invention, a sustainable aerobic methane degradation media 260 is interposed between the methane source and the surface 230 or point at which the methane is released into the atmosphere. According to one embodiment of the present invention, methanotrophic bacteria from the native soil are added to the media 260. In one embodiment of the present invention, native methanotrophs 250 are extracted from the native soil and/or grown in a liquid growth medium and reintroduced into the media at a concentration sufficient to mitigate the methane.

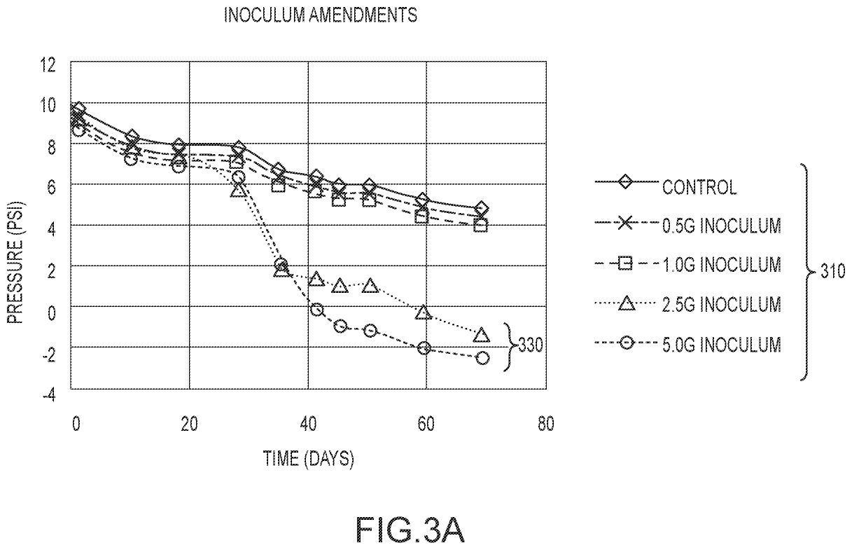

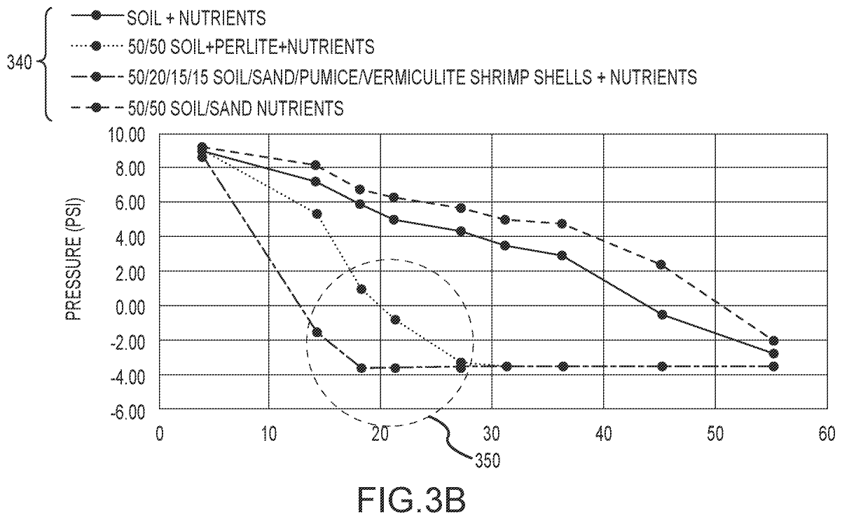

FIG. 3A presents data of increased methane biodegradation as a result of increased presence of methanotrophs. Tests show that a minimum quantity of methanotrophs is required to activate methane biodegradation. FIG. 3 presents data showing methane biodegradation from methanotrophs gained from a methane release site (native soil) and added back to site lacking (sufficient) methanotrophs in varying quantities 310 resulting in various doses of soil (0.5 g-1.35% by weight; 1 gram-2.7%; 2.5 g-6.8%; 5 g-13.5%). A nutrient solution was also applied to the media. The results demonstrate that a minimum 330, in this scenario, of between 2.7% and 6.8% dose of soil (methanotrophs) from the methane release site was required to stimulate methane biodegradation (as shown by a lower methane pressure). This data demonstrated that a threshold of methanotrophs is needed to create a sustainable environment for methane biodegradation.

As discussed herein the concentration and ratio of matrix components within the methane biodegradation media is an important factor the methane biodegradation. FIG. 3B presents data representative to a landfill methane release site in which various matrix configurations 340 result in varied methane biodegradation. The graphic shows results (average of duplicate incubations) wherein the landfill methane release site soil was amended with nutrients alone and with nutrients in combination with various drainage materials including 50% by volume sand, 50% by volume perlite, and a mixture of perlite, sand, pumice, vermiculite, and ground shrimp shells. To simulate a precipitation event the test area was spiked with a relatively large volume of water to saturate the media to the extent that a layer of free water gathered at the bottom of the test site. The results show that the addition of specific drainage materials to the soil was required for effective methane biodegradation 350. Notably, either perlite alone (50% by volume) or a 50/20/15/15 mixture of soil, sand, pumice, and vermiculite with ground shrimp shells added as a long-term source of ammonia nutrient produced excellent results.

As one of reasonable skill will appreciate, methanotrophic bacteria require certain environmental conditions by which to oxidize methane. For example, oxygen must be present. Moisture is also a key factor in supporting sustained aerobic methane degradation. Too little or too much moisture can be problematic and lead to incomplete and/or inadequate degradation.

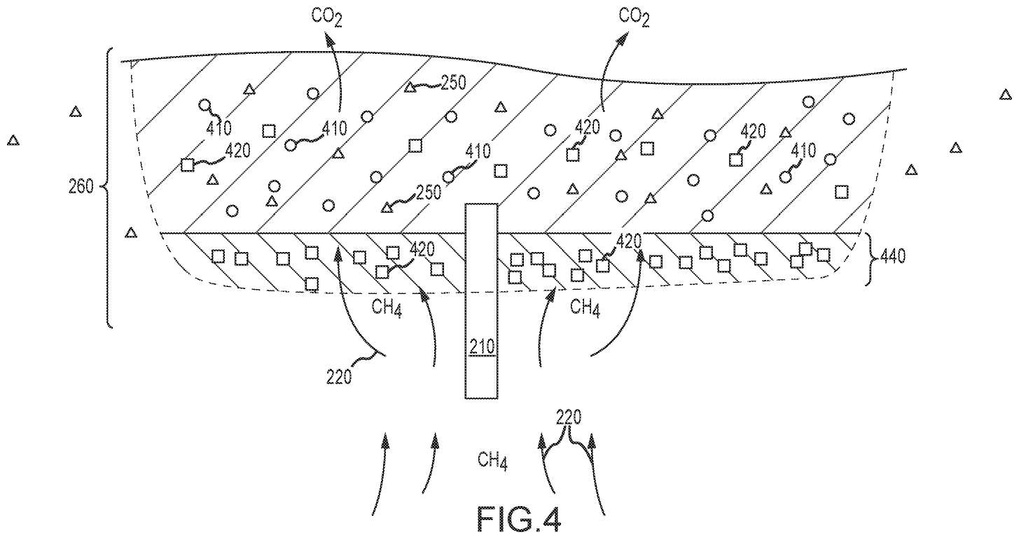

As illustrated in FIG. 4, the methane degradation media 260 of the present invention includes an inorganic matrix 450 having a moisture management component 340 and a drainage component 420. The moisture management component 410 of the present invention's inorganic matrix ensures methanotrophs present in the media are provided adequate moisture for sufficient methane oxidation. Examples of the moisture management component 410 include vermiculite, pumice, and the like.

As a methane degradation media formulated for dry conditions may not support or sustain methane biodegradation under wet conditions and vice versa, one embodiment of the present invention, formulates media for different conditions that are applied in separate layers. In one embodiment a drainage layer 440 of highly concentrated drainage components 420 underlies the remaining matrix.

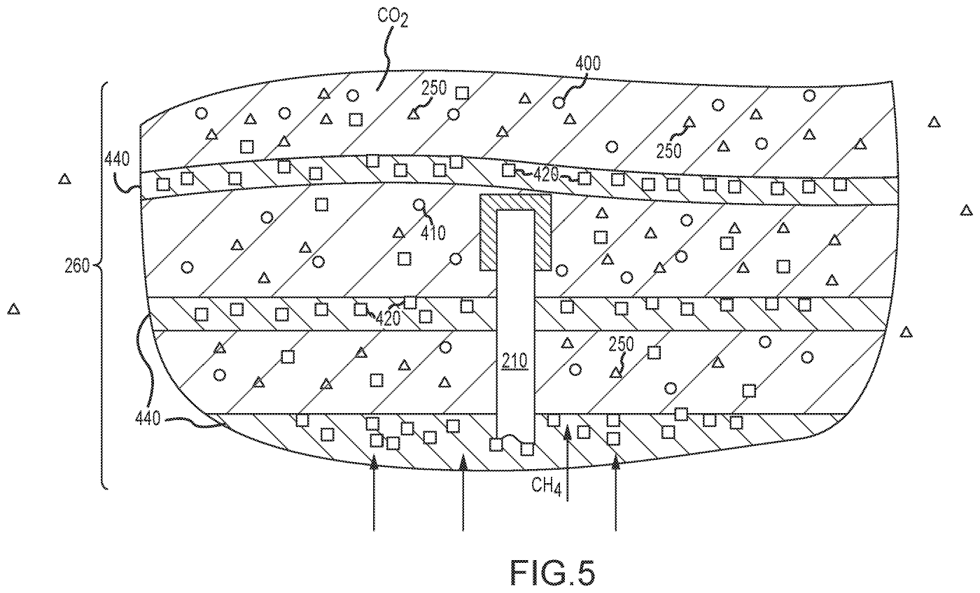

Test results show that moisture retaining materials with a high cation exchange capacity including vermiculite and pumice can scavenge needed nutrients and reduce methane biodegradation rates. Magnesium and/or calcium are added to the methane to block nutrient uptake by the cation exchange sites. In another embodiment of the present invention, moisture retaining media are applied in separate layers as can be seen in FIG. 5.

In arid conditions the inorganic matrix includes a higher ratio of a moisture management component 410 than would be normally found in the native soil. For example, in arid conditions the native soil may not include a means necessary to retain moisture sufficient to sustain the identified concentration of methanotrophs needed to degrade the local methane flow rate. As the methanotrophs are supplemented so too must be the ability of the soil, the matrix, to retain moisture be enhanced. According to one embodiment of the present invention the ratio of the moisture management component as compared to the drainage component is configured to create a sustainable methane biodegradation environment. This configuration is based one or more factors including the localized methane flow rate, the concentration of methanotrophs as compared to the native soil, and the local environment.

In some instances, the ratio of the moisture management component to the drainage component may be 2:1. While in other environments it may be 3:1. And when the methane source is found in area of high precipitation the ratio may be 1:2. The ratio of the moisture management component to the drainage component will vary based on the local environment, the soil type, and the methane flow rate.

As presented above, one embodiment of the present invention can include a ratio of the moisture management component to the drainage component of 1:2, 1:4 or the like. Methanotrophic bacteria require a certain degree of moisture to survive and conduct methane oxidation. Oxidation requires oxygen and the ability to have gas exchange through the media. Water saturation and soil compaction preclude such an exchange. The drainage component ensures that the ability for gas exchange to occur is preserved. Examples of the drainage component include perlite, pumice, sand, and gravel. As implied above, oxygen unavailability can limit the extent and rate of methane biodegradation especially when the flow of methane is high. In one instance actively aerating methane biodegradation media includes pumping air or oxygen into the methane degradation media. In another embodiment, the methane degradation media is passively aerated using intake piping positioned below the inorganic degradation media. As the methane flows upward through the media and methanotrophs convert methane to carbon dioxide generated heat and the gas flow induces air to be introduced below and into the methane biodegrading media. The added oxygen promotes additional degradation.

Consider further a localized methane source in a tropical environment or one which receives excessive precipitation as shown in FIG. 5. Again, a manmade methane source 210 has disturbed the ecosystem enabling a higher-than-normal methane flow rate. While the methanotrophs have sufficient moisture to survive, the ability to conduct methane oxidation is precluded by moisture saturation. In such an instance the matrix imposed near the methane source possesses a higher ratio of the drainage component 420 to disperse excess moisture. Material such perlite, sand, gravel, and the like can direct moisture away from the methane source and enabling the methanotrophs to oxidize methane. The configuration of the matrix may also include multiple drainage layers 440 enabling excess moisture to be dispersed.

In addition to methanotrophs, the matrix, as part of the media, is further combined with long-lasting inorganic nutrients including phosphate minerals, chitin-rich materials, and cationic nutrients including ammonia and trace metals. Calcium carbonate can be added for pH control. These nutrients can be loaded onto the high cation exchange capacity of vermiculite and pumice. In other embodiments the nutrients can be added in addition to or in lieu of water.

In another embodiment of the present invention, the surface of one or more components of the inorganic media is coated with soil containing native methanotrophs.

Methane is lighter (lower density) than air. Accordingly, it migrates through soil until it is released into the atmosphere. Localized methane biodegradation is enhanced, according to one embodiment of the present invention, by structuring the methane biodegradation media. In one instance, a laterally dispersed and highly concentrated layer of the drainage component such as perlite is positioned below a mixture of the inorganic matrix and methanotrophs. Recall that the drainage component is characterized as being highly permeable with low water retention helping to prevent soil compaction. As methane rises from a local source, the drainage component layer disperses methane within the porosity of the media and away from the source much like it disperses infiltrating precipitation enabling a larger exchange of gases than would be possible if the methane was allowed to naturally percolate through the soil.

In another embodiment, the media is generated by supplementing soil native to the area of the methane release with a drainage component in wet environments, with a moisture management component in dry environments, or combinations of a drainage component and moisture management component. The ratio of moisture drainage and moisture management components can be dependent on the soil type and precipitation levels. For example, a sandy soil in an arid environment would require a larger percentage of moisture management components. A clay soil in a wet environment would require a larger percentage of moisture drainage components.

In another embodiment, the methane biodegradation media of the present invention is inserted into the casing of an oil and gas well or vent pipes on top of landfills to intercept upward leaking methane. In such an instance methane biodegradation media is loaded inside perforated PVC or similar piping. A highly transmissive mixing zone at the base of the piping (near the methane source) allows upward migrating methane to mix with air that is introduced from the surface through tubing. The loaded methane biodegradation media pipe is lowered and secured into the well or vent. Alternatively, the methane biodegradation media is constructed directly inside the well or well casing. In such an instance highly, transmissive material is poured into the well to create the air and methane mixing zone followed by the methane biodegradation media.

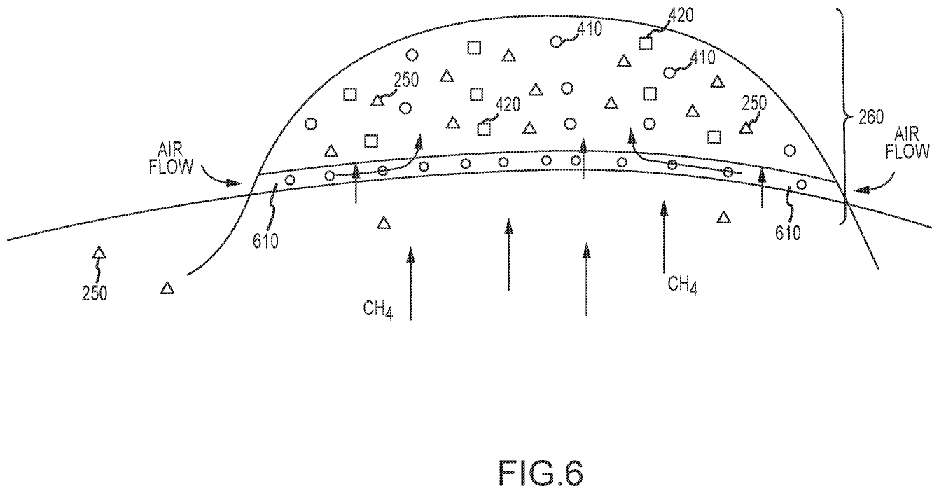

One embodiment of the inorganic matrix structure of present invention employs a mounded configuration or format as shown in FIG. 6. While illustrative of the advantages of the present invention, one of reasonable skill in the relevant art will recognize other configurations may be utilized without departing from the scope and intent of the present invention. In a mounded configuration heat generated during methane biodegradation heats the media relative to the ambient air temperature resulting in the upward flow of gases in the methane biodegradation media, pulling air directly into the media through highly permeable/transmissible layers of the moisture management component 410 and the drainage component 420. In other embodiments perforated air intake pipes 610 are installed in or below the media. Alternatively, the inorganic matrix structure can be configured as a flat or nearly flat layer of an appropriate thickness promoting passive aeration over a large area such as a landfill.

One approach for passive methane biodegradation includes placing sustainable aerobic methane biodegradation media in a mounded configuration promoting upward gas flow enhanced by the presence of, and incorporation of, a perforated air intake pipe. Layer(s) of highly transmissible materials are added above the methane source to provide a pathway for air to enter the media and disperse throughout the matrix.

Cold temperatures bring significant challenges in sustaining methane biodegradation using thin, flat, or nearly flat near surface methane biodegradation media formats. Cold temperatures result in decreased methane biodegradation rates and frozen water which fills the porosity of the matrix blocking gas transmission. In addition, flat thin formats encourage saturation of the methane biodegradation media porosity with precipitation and surface drainage decreasing gas exchange.

According to one embodiment of the present invention, the drainage component is combined with material having insulating properties including but not limited to vermiculite, pumice, perlite, fiberglass insulation, and the like. These materials can be placed as layer(s) in the matrix and/or throughout the media to trap heat generated during methane biodegradation. The methane biodegradation media and insulating layer can be modified to include elements to enhance the drainage of infiltrating precipitation precluding water saturation of the insulating material.

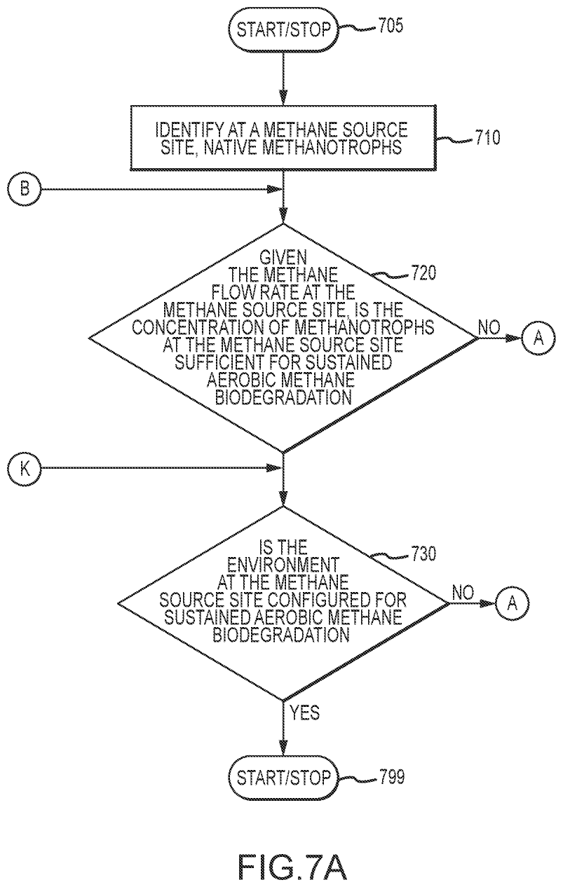

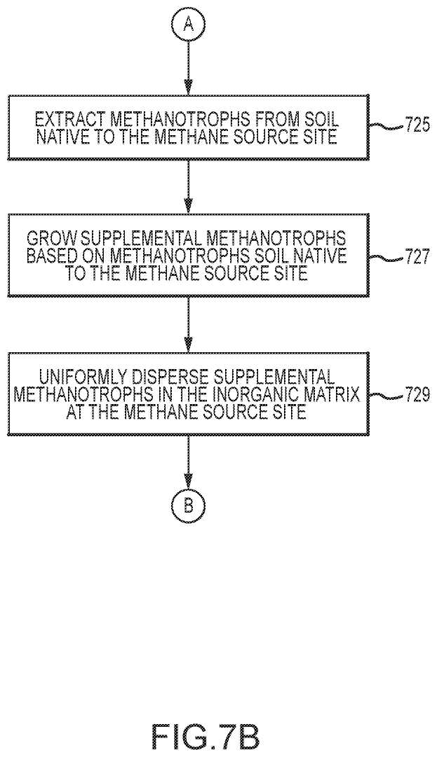

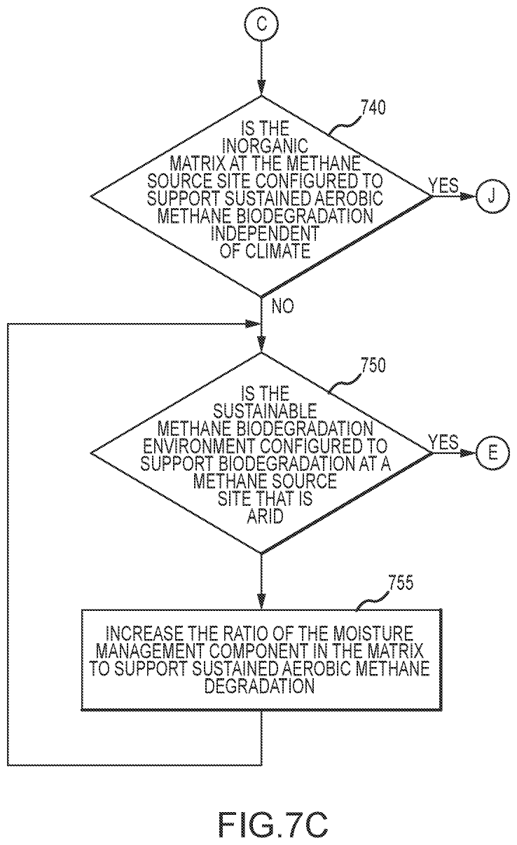

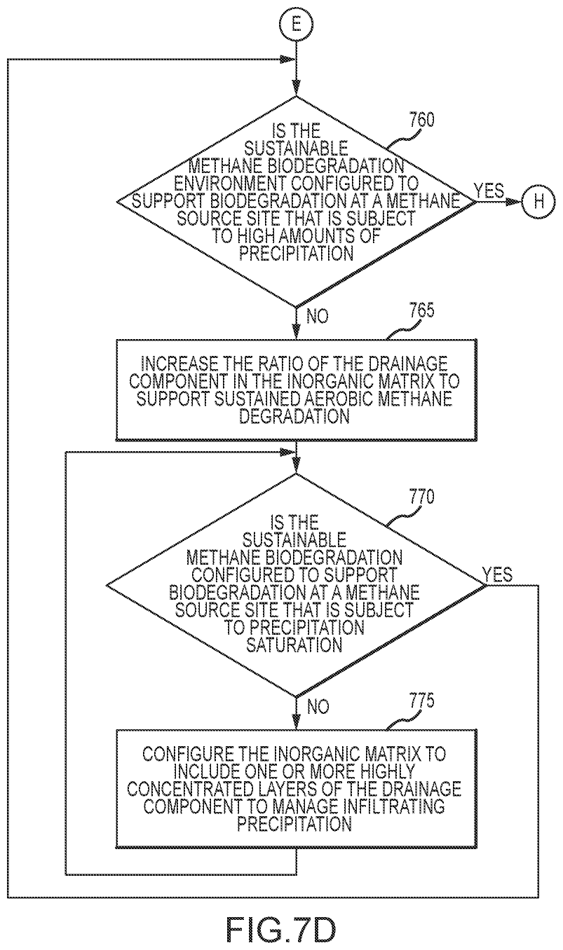

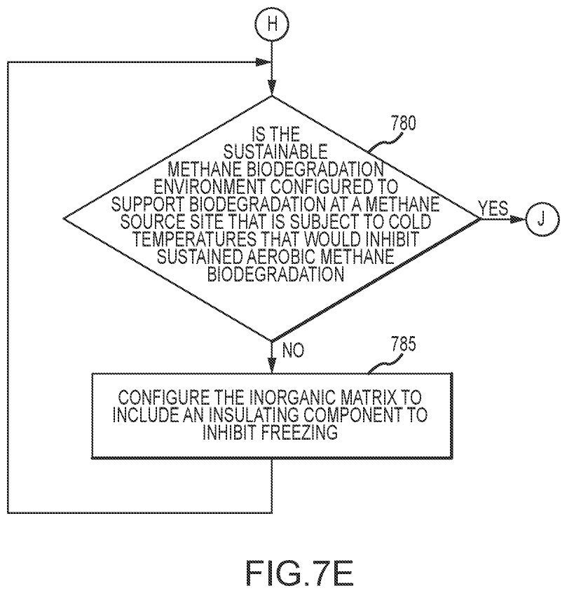

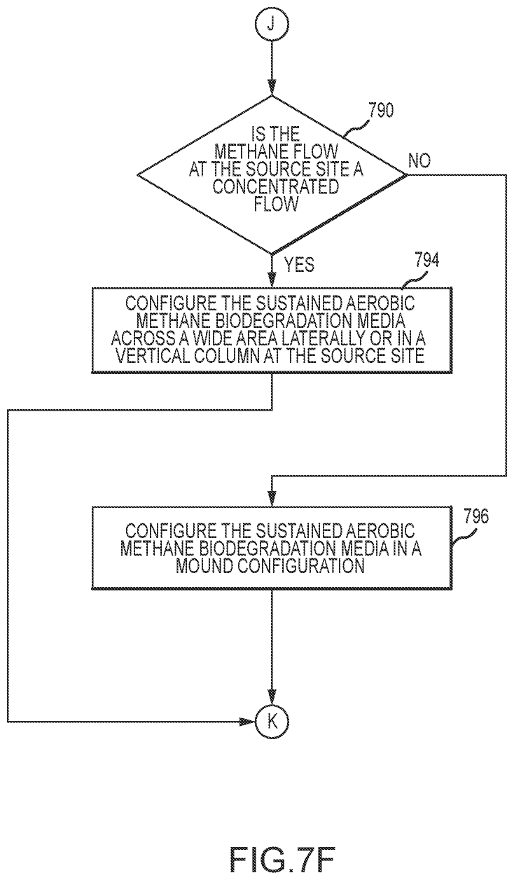

FIGS. 7A-7F depict a flowchart of a methodology which may be used to aerobically biodegrade methane. In the following description, it will be understood that each block of the flowchart illustrations, and combinations of blocks in the flowchart illustrations, can be implemented by hardware, firmware, or computer program instructions. When implemented by a computer the instructions may be loaded onto a machine or other programmable apparatus such that the instructions that execute on the machine or other programmable apparatus create means for implementing the functions specified in the flowchart block or blocks. These computer program instructions may also be stored in a computer-readable memory that can direct a computer or other programmable apparatus to function in a particular manner such that the instructions stored in the computer-readable memory produce an article of manufacture including instruction means that implement the function specified in the flowchart block or blocks. The computer program instructions may also be loaded onto a computer or other programmable apparatus to cause a series of operational steps to be performed in the computer or on the other programmable apparatus to produce a computer implemented process such that the instructions that execute on the computer or other programmable apparatus provide steps for implementing the functions specified in the flowchart block or blocks.

Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware-based computer systems that perform the specified functions or steps, or combinations of special purpose hardware and computer instructions.

The flowchart shown in FIGS. 7A-7F present one methodology for sustained aerobic methane biodegradation, according to one embodiment of the present invention. The process begins 705 with identifying 710, at a methane source site, methanotrophs native to the local soil. Based on the methane flow rate at the source site, a concentration of methanotrophs native to the soil at the methane source site sufficient to sustain aerobic biodegradation is determined 720.

In the instance that the native concentration of methanotrophs is insufficient for sustained aerobic biodegradation of the source site's methane flow rate, methanotrophs, from the native soil, are extracted 725 and isolated. Supplemental methanotrophs are thereafter grown 727 or replicated based on the extracted native methanotrophs creating a complementary methanotroph supply. These supplemental methanotrophs are thereafter uniformly dispersed 729 in the inorganic matrix of a sustained aerobic methane biodegradation media raising the local concentration of methanotrophs to a measure sufficient for sustained biodegradation of methane at the source site's methane flow rate.

With sufficient methanotrophs resident in the biodegradation media, an inquiry 730 is made as to the environment in which biodegradation will take place. As one of reasonable skill in the relevant art will appreciate, the native soil and environment surrounding a methane source site may not be conducive for sustained aerobic methane biodegradation. In this example, the process first examines whether the inorganic matrix at the source site is configured to support sustained aerobic methane biodegradation independent of climate 740. Said differently, the inquiry is whether the media and matrix can operate in the local environment to biodegrade the methane on a sustained basis, regardless of the local weather conditions.

To ascertain the correct formation of the inorganic matrix to support the methane biodegradation, a series of inquiries are made. The first, in this example, is whether the sustainable methane biodegradation environment is configured to support biodegradation at a methane source site that is arid 750. Arid or dry conditions may not support methane biodegradation. The present invention configures the inorganic matrix to control the moisture and ensure that conditions are present to support a concentration of methanotrophs necessary for methane biodegradation at the source site. In an arid environment the ratio of moisture management component of the inorganic matrix is increased/modified 755 to maintain adequate moisture for methanotroph survival and activity.

If the source site is not arid the inquiry continues by exploring whether the methane source site is subject to high amounts of precipitation 760. While methanotrophs require sufficient moisture to survive, methane biodegradation is an aerobic process. Water saturation of the soil can also inhibit methane biodegradation. To rectify this challenge the ratio of the drainage component of the inorganic matrix is increased/modified 765.

When the environment is subjected to water saturation 770, the structure of the matrix itself can be altered. In one embodiment of the present invention, one or more layers 775 of highly concentrated drainage component can included to manage infiltrating precipitation. The layers can be sloped to facilitate drainage away from the biodegradation media/source site to ensure that aerobic conditions are maintained enabling sustained methane biodegradation.

While moisture management is an important aspect of the present invention, temperature also is a factor to be considered 780. In regions in which the ambient temperature falls below freezing (0 degrees Celsius), the moisture maintained for methanotrophs survival can freeze and inhibit sustained methane biodegradation. Depending on the intensity of the climate, the depth at which soil freezes varies. Commonly known as the frost line, it represents a depth of soil that below which the ground water remains liquid. Aerobic processes and indeed methane oxidation produces a certain amount of heat as a byproduct. The present invention, in one embodiment, configures the inorganic matrix to include an insulating 785 component to inhibit freezing by capturing heat produced by bio degradation.

One of reasonable skill in the relevant art will appreciate that while moisture and temperature are important factors to consider in creating a sustainable methane biodegradation environment, other aspects may also need to be managed. The present invention configures the matrix based on the locality of methane source site and the methane flow rate to create a sustainable methane biodegradation environment.

The process of FIG. 7 also considers structure in forming an appropriate sustainable aerobic methane biodegradation environment. In the instance of the methane flow being concentrated 790 at one location, the present invention configures the sustained aerobic methane biodegradation media in a vertical column 799. Various layers, concentration, and ratios can be used and implemented to capture and mitigate the methane. Wells, and in particular, oil wells, form a concentrated methane flow at the methane source. In one embodiment of the present invention, the well site can be excavated, and the methane biodegradation media placed in a vertical configuration over the column of methane. Note that one approach used by the present invention is not to cap the source but rather biodegrade the methane in a sustainable manner. Capping or sealing the well or even the column may cause the methane to be diverted to a different location. In the present invention the flow and column of methane is not diverted. The methane biodegradation media is placed in the natural path of the methane enabling the media to oxidize the methane as it makes it way to the surface.

When the methane source is widely dispersed, such as in a landfill or the like, a lateral layer of aerobic methane biodegradation media that can sustain methane biodegradation under diverse and fluctuating conditions is installed. In some instances, a mound configuration 796 is used to channel the methane through additional media to enhance the efficiencies of methane oxidation.

One or more embodiments of the present invention present a sustained aerobic methane biodegradation and application approaches that result in highly effective and sustainable methane biodegradation around methane source sites, with the media and the configuration for which the media is applied in the ground around the sites being arranged to achieve active methane biodegradation for long periods of time with limited or no maintenance under diverse and fluctuating environmental conditions including temperature and moisture levels. The biodegradation media of the present invention includes different solid-phase materials that maximize contact between methane and methanotrophs, different inoculum sources, minerals to supply long-lasting sources of nutrients, and methods to develop these inoculum sources. Enriching microbial inocula obtained from areas under similar conditions to where the biodegradation media will be applied results in more efficient methane biodegradation under site specific conditions. Furthermore, enriching (growing) inocula on the same biodegradation media that will be applied in the field results in more effective methane biodegradation. Long lasting nutrient sources, including mineral sources of nutrients can be used to support methane oxidation for extended periods. An effective and permanent biodegradation media support mixed with long-lasting mineral sources of nutrients inoculated as described above when placed in the ground in a format that precludes the media from drying out or being overly saturated allows for rapid sustainable methane biodegradation.

While there have been described above the principles of the present invention in conjunction with a sustained aerobic methane biodegradation, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features that are already known per se, and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves the right to formulate new claims to such features and/or combinations of such features during the prosecution of the present application or of any further application derived therefrom.

1. A sustainable aerobic methane biodegradation media, comprising:
methanotrophs native to a methane source at a concentration sufficient for methane biodegradation based on a methane flow rate; and
an inorganic matrix sufficient for sustained methane biodegradation wherein the inorganic matrix includes an inorganic matrix structure configured to manage moisture and create a sustainable methane biodegradation environment.

2. The sustainable aerobic methane biodegradation media of claim 1, wherein the media includes methanotrophic bacteria based on native soil extracted at the methane source.

3. The sustainable aerobic methane biodegradation media of claim 1, wherein the media includes methanotrophs replicated from native soil at the methane source and wherein the methanotrophs replicated from native soil are uniformly dispersed throughout the matrix.

4. (canceled)

5. The sustainable aerobic methane biodegradation media of claim 1, wherein the inorganic matrix includes a moisture management component configured to manage moisture content and a drainage component configured to manage infiltrating precipitation.

6. The sustainable aerobic methane biodegradation media of claim 5, wherein the inorganic matrix structure is configured based on the methane flow rate at the methane source and configured to provide sufficient surface area interaction between the methanotrophs and methane at the methane source based on the methane flow rate.

7. (canceled)

8. (canceled)

9. The sustainable aerobic methane biodegradation media of claim 5, wherein the moisture management component is selected from the group consisting of vermiculate, peat, perlite, and sawdust.

10. The sustainable aerobic methane biodegradation media of claim 5, wherein the drainage component is selected from the group consisting of pumice, sand, perlite, and gravel.

11. The sustainable aerobic methane biodegradation media of claim 5, wherein the inorganic matrix structure is a vertical layered structure having one or more successive layers of the moisture management component and the drainage component.

12. The sustainable aerobic methane biodegradation media of claim 5, wherein responsive to the methane source being in an arid environment, the inorganic matrix structure includes a base layer of a highly concentrated layer of a moisture retaining material selected from the group consisting of vermiculate, peat, pumice, and sawdust.

13. The sustainable aerobic methane biodegradation media of claim 5, wherein responsive to the to the methane source being in a tropical environment, the inorganic matrix structure includes a sloped drainage layer of a material configured to manage infiltrating precipitation selected from the group consisting of pumice, sand, perlite, and gravel.

14. The sustainable aerobic methane biodegradation media of claim 5, wherein responsive to the to the methane source being in being in an arid environment, a ratio of the moisture management component to the drainage component is at least 2:1.

15. The sustainable aerobic methane biodegradation media of claim 5, wherein responsive to the methane source being in a tropical environment, a ratio of the drainage component to the moisture management component is at least 2:1.

16. (canceled)

17. The sustainable aerobic methane biodegradation media of claim 1, wherein the inorganic matrix further comprises carbonate rock configured for pH control.

18. (canceled)

19. The sustainable aerobic methane biodegradation media of claim 1, wherein the moisture management component includes materials with a plurality of cation exchange sites and wherein the plurality of cation exchange sites are bound with added nutrient cations and wherein the inorganic matrix further comprises magnesium and/or calcium configured to block nutrient uptake by the plurality of cation exchange sites.

20. (canceled)

21. The sustainable aerobic methane biodegradation media of claim 1, wherein the inorganic matrix further comprises added cationic nutrients sufficient for methane biodegradation.

22. The sustainable aerobic methane biodegradation media of claim 1, wherein the inorganic matrix further comprises a pH buffer configured to control pH of the biodegradation media.

23. The sustainable aerobic methane biodegradation media of claim 5, further comprises a nutrient nitrogen concentration based on moisture content of the inorganic matrix.

24. The sustainable aerobic methane biodegradation media of claim 23, wherein the nutrient nitrogen is an aqueous nutrient nitrogen solution.

25. The sustainable aerobic methane biodegradation media of claim 1, wherein the inorganic matrix further comprises a phosphorus mineral source.

26. A method for sustained aerobic methane biodegradation, comprising:
identifying, at a methane source site, native methanotrophs;
determining, at the methane source site, a concentration of native methanotrophs sufficient for methane biodegradation based on a methane flow rate; and
configuring, at the methane source site, an inorganic matrix for sustained methane biodegradation wherein the inorganic matrix includes an inorganic matrix structure configured to manage moisture content, manage moisture drainage, and create a sustainable methane biodegradation environment.

27. The method for sustained aerobic methane biodegradation according to claim 26, further comprising extracting methanotrophs from the methane source site and thereafter growing methanotrophic bacteria and supplementing methanotrophs at the methane source site with grown methanotrophic bacteria.

28. (canceled)

29. (canceled)

30. The method for sustained aerobic methane biodegradation according to claim 26, further comprising configuring a moisture management component of the inorganic matrix to manage moisture content and configuring a drainage component of the inorganic matrix to manage infiltrating precipitation.

31. The method for sustained aerobic methane biodegradation according to claim 30, further comprising configuring the inorganic matrix structure based on the methane flow rate at the methane source.

32. The method for sustained aerobic methane biodegradation according to claim 31, further comprising configuring the inorganic matrix structure to provide sufficient surface area interaction between methanotrophs and methane at the methane source based on the methane flow rate.

33. The method for sustained aerobic methane biodegradation according to claim 30, further comprising layering the inorganic matrix structure vertically having one or more successive layers of the moisture management component and the drainage component.

34. The method for sustained aerobic methane biodegradation according to claim 33, wherein responsive to the methane source being in an arid environment, configuring the inorganic matrix structure with a base of a highly concentrated layer of a moisture retaining material selected from the group consisting of vermiculate, peat, pumice, and sawdust.

35. The method for sustained aerobic methane biodegradation according to claim 30, wherein responsive to the to the methane source being in a tropical environment, configuring the inorganic matrix structure with a sloped drainage layer of a material configured to manage infiltrating precipitation selected from the group consisting of pumice, sand, perlite, and gravel.

36. The method for sustained aerobic methane biodegradation according to claim 30, wherein responsive to the to the methane source being in being in an arid environment, configuring the inorganic matrix to have a ratio of the moisture management component to the drainage component is at least 2:1.

37. The method for sustained aerobic methane biodegradation according to claim 30, wherein responsive to the methane source being in a tropical environment, configuring the inorganic matrix to have a ratio of the drainage component to the moisture management component is at least 2:1.

38. (canceled)

39. The method for sustained aerobic methane biodegradation according to claim 26, further comprising binding one or more cation exchange sites of the moisture management component with cations.
